(19) United States
(12) Patent Application Publication
(10) Pub. No.: US 2022/0323567 A1
(43) Pub. Date: Oct. 13, 2022

(54) COMBINATION VACCINE FOR INTRADERMAL ADMINISTRATION

(71) Applicant: INTERVET INC., Madison, NJ (US)

(72) Inventors: Theodorus Jansen, Boxmeer (NL); Melanie SNO, Boxmeer (NL); Martin PIEST, Boxmeer (NL)

(21) Appl. No.: 17/641,031

(22) PCT Filed: Sep. 11, 2020
(86) PCT No.: PCT/EP2020/075454

§ 371 Date: Mar. 7, 2022

(30) Foreign Application Priority Data
Sep. 12, 2019 (EP) 19196890.8

(51) Int. Cl. A61K 39/12; A61K 39/02; A61K 47/02; A61K 47/22; A61K 47/06; A61P 31/20; A61P 31/04

(52) U.S. Cl. CPC A61K 39/12; A61K 39/02; A61K 47/02; A61K 47/22; A61K 47/06; A61P 31/20; A61P 31/04; A61K 2039/70

(57) ABSTRACT

The present invention relates to the field of veterinary vaccinology, namely to combination vaccines for swine. In particular the invention relates to a combination vaccine for protection against a pathogenic infection with porcine circo vims type 2 (PCV2) and Mycoplasma hyopneumoniae (Mhyo) comprising non-replicating immunogen of PCV2 and non-replicating immunogen of Mhyo. The vaccine is characterized in that it is an oil-in-water emulsion comprising squalane, vitamin E-acetate and silica. In another embodiment, the invention relates to a combination vaccine for protection against a pathogenic infection with PCV2 and Mhyo by intradermal administration.

### GENERAL FIELD OF THE INVENTION

The present invention relates to the field of veterinary vaccinology, namely to combination vaccines for swine. In particular the invention relates to a combination vaccine for protection against a pathogenic infection with porcine circo virus type 2 (PCV2) and Mycoplasma hyopneumoniae (Mhyo) comprising non-replicating immunogen of PCV2 and non-replicating immunogen of Mhyo. The vaccine is characterized in that it is an oil-in-water emulsion comprising squalane, vitamin E-acetate and silica. In another embodiment, the invention relates to a combination vaccine for protection against a pathogenic infection with PCV2 and Mhyo by intradermal administration.

### BACKGROUND OF THE INVENTION

Intensive swine farming today, relies heavily on veterinary medical products to keep animals healthy and to allow an economic operation. Next to optimisation of the feed and of farm management systems, a variety of treatments are regularly used: pharmaceuticals such as hormones or antibiotics, and vaccination against bacterial- or viral pathogens. Some of the most prominent diseases affecting swine from a young age onwards are caused by bacteria such as Mycoplasma hyopneumoniae and by viruses such as porcine circovirus type 2.

Mycoplasma hyopneumoniae (Mhyo) is the primary agent causing (porcine) enzootic pneumonia, a chronic respiratory disease in swine, occurring worldwide. Especially young piglets are vulnerable to this highly contagious disease. The bacterium is relatively small, lacks a cell wall, and belongs to the genus Mollicutes. These bacteria live a parasitic lifestyle on- or in host cells. Pulmonary disease from Mhyo is largely an immune-mediated pathology leading to consolidated pneumonia. The bacterium colonizes and damages the pulmonary ciliated epithelium, leading to loss of cilliary activity. Depending on housing conditions and environmental stress, the most problematic consequence of this disease is that it predisposes for different secondary infections of the porcine respiratory system by other bacterial- and viral pathogens. This gives rise to the so called: Porcine Respiratory Disease Complex (PRDC), displaying severe lung lesions. Next to discomfort to the animal, enzootic pneumonia and PRDC cause important economic losses to the swine industry due to reduced performance in growth rate and feed conversion ratio, as well as through costs for veterinary care and antibiotics use.

Porcine circovirus type 2 (PCV2) is linked to the post-weaning multisystemic wasting syndrome (PMWS) observed in young pigs. The clinical signs and pathology were published in 1996, and include progressive wasting, dyspnoea, tachypnoea, and occasionally icterus and jaundice. The new agent was called PCV2 as being different from the known PCV, that was a natural contaminant of PK-15 cells. PCV2 is a very small non-enveloped virus of the Circovirus genus. It contains a circular single stranded DNA genome with two major genes. The ORF2 gene encodes the viral capsid protein of about 233 amino acids. Recombinantly expressed PCV2 ORF2 proteins form virus like particles which are highly effective as a subunit vaccine.

Against Mhyo a variety of commercial vaccines exists, and these are routinely used in the majority of commercial swine farming operations. Generally, these vaccines comprise non-replicating immunogens such as subunit proteins and/or bacterins (i.e. killed bacteria, intact or not), which are typically administered by parenteral injection. Some examples are: RespiSure® (Zoetis), Ingelvac® M. hyo (Boehringer Ingelheim), and M+Pac® (Merck Animal Health).

A conventional vaccine to prophylactically treat animals, in particular pigs, against an infection with PCV2, may be based on whole inactivated PCV2 virus as (non-replicating) immunogen. Also, in the art it has been shown that the ORF2 encoded capsid protein (e.g. when recombinantly expressed) is suitable as a subunit immunogen of PCV2 for use in an adequate vaccine. This can be understood since this subunit in the body shows up the same way as the virus itself (it forms virus-like particles), essentially differing only in the fact that the DNA and non-structural proteins are not present inside the capsid. In the art several vaccines against PCV2 are commercially available. Porcilis® PCV (available from MSD Animal Health, Boxmeer, The Netherlands) is a vaccine for protection of pigs against porcine circo virus type 2, for use in pigs from three weeks and older. When given as a two-shot (two dose) vaccine, the duration of immunity (DOI) is 22 weeks, almost completely covering the fattening period of pigs. Ingelvac CircoFlex® (available from Boehringer Ingelheim, Ingelheim) is a vaccine for protection of pigs against porcine circo virus type 2, for use in pigs from two weeks and older. It is registered as a one-shot (one dose) vaccine only. Circovac® (available from Merial, Lyon, France) is a vaccine for protection of pigs against porcine circo virus type 2, for use in pigs three weeks and older. Suvaxyn® PCV (available from Zoetis, Capelle a/d IJssel, The Netherlands) is a vaccine for protection of pigs against porcine circo virus type 2, for use in pigs from three weeks and older. Other PCV2 vaccines are described for example in WO2007/028823, WO 2007/094893 and WO2008/076915.

To limit stress to the animals and cost and labour for the caretakers, some swine vaccines have been prepared as combination vaccine. Examples are: Ingelvac CircoFLEX and Ingelvac MycoFLEX (Boehringer), which can be mixed shortly before administration, Fostera® PCV MH (Zoetis) and Porcilis® PCV MHyo (MSD Animal Health), which combine antigens from PCV2 and Mhyo.

An important component of vaccines comprising non-replicating immunogens is an adjuvant. This provides an immune-stimulation for the non-replicating immunogen, which would otherwise not be immunogenic. This will trigger different routes of the immune system, the basic mechanisms are not well understood. In veterinary vaccines, a great variety of compounds can be used as adjuvant, for example: mineral oil e.g. Bayol® or Markol®, Montanide® or paraffin oil; non-mineral oil such as squalene, squalane, or vegetable oils, e.g. ethyl-oleate; aluminium salts, e.g. aluminium-hydroxide, or aluminium-phosphate; peptides such as dimethylglycine, or tuftsin; bacterial cell-wall components, such as lipid A and muramyldipeptide; (synthetic) polymers, such as pluronics, dextranes, carbomeres, pyran, or saponin; cytokines; and stimulators of toll-like receptors such as immunostimulatory oligodeoxynucleotides containing non-methylated CpG groups; etc.

The main problem to overcome in making adjuvated combination vaccines is to prevent an interaction between the various vaccine components that would negatively influence the immune response or the vaccine's safety or stability. Such interaction may for instance occur between the immunogens themselves, e.g. because some are quite crude products, such as the bacterins of Mhyo. However, for PCV2 and Mhyo, the art has already shown that non-replicating immunogens of various types of these pathogens can be combined in an effective vaccine. However, the adjuvant may interfere with, or even damage a vaccine immunogen. This is also recognised by the registration authorities providing marketing authorisations, for example: the USDA enforces regulation 9CFR 113.35 for detection of virucidal activity in an inactivated vaccine comprising a live virus. Next to this, the particular route of administration may have significant impact on the safety of an adjuvant composition: an adjuvant may be safe when administered intramuscularly, but lead to unacceptable safety problems when administered subcutaneously.

These potential problems in the development of complex combination vaccines are generally recognised; see for example a publication from the EMEA: “Note for guidance: requirements for combined veterinary products” (EMEA, 2000, CVMP/IWP/52/97-FINAL); and a publication from the U.S. Department of Health and Human Services, Food and Drug Administration, Center for Biologies Evaluation and Research, from April 1997: “Guidance for Industry, for the evaluation of combination vaccines for preventable diseases: Production, Testing and Clinical Studies”, Docket No. 97N-0029.

Both these publications warn for the effects of interferences on the efficacy and safety of a vaccine, when combining immunogens and adjuvants.

It is thus difficult to develop a combination vaccine which induces an effective immune-response against complex combinations of immunogens relating to multiple species of pathogens. Further the combination vaccine should be safe upon use in animals, i.e. not produce significant side reactions such as fever, local swelling, loss of appetite, etc. Also more practical properties are relevant: the combination vaccine should ideally be capable of economic production, be sufficiently stable during formulation and storage, and allow potency testing methods for each immunogen, in the presence of the other immunogens.

All in all, it is commonly known that combined vaccination against multiple pathogens is not straightforward and requires experimentation to determine safety and efficacy, in particular when the combined vaccination is an adjuvanted combination vaccine.

Thus, there is a need to overcome one or more disadvantages in the prior art, and to provide an effective and safe combination vaccine against diseases associated with infections by PCV2 and Mhyo.

In the vaccination of large numbers of animals, it is of importance to limit stress for both the animals and the people who vaccinate them. Further, vaccination via the conventional intramuscular route is often associated with pain and stress for the animals and is associated with an increased risk of side reactions and infections. One possibility to overcome the problems associated with intramuscular administration is vaccination by administration into the dermis, also referred to as intradermal administration. Intradermal vaccination allows for a wider range of administration sites in the animal, providing increased flexibility for the user. This is particularly useful when vaccinating large groups of pigs as it allows quick and non-invasive application, creating less stress for both pigs and the administrator.

The systemic and respiratory local immune response induced by the intradermal administration of a commercial inactivated Mycoplasma hyopneumoniae whole-cell vaccine (Porcilis® MHYO ID ONCE -MSD Animal Health) in comparison with two commercial vaccines administered via the intramuscular route and a negative control was described by P. Martelli et al., Vet Microbiol.

2014; 168(2-4):357-64, showing that the intradermal administration of an adjuvanted Mhyo bacterin induces both systemic and mucosal immune responses.

In particular, intradermal administration has the advantage that it can be performed by needleless vaccination devices, such as the IDAL® vaccinator (available from MSD Animal Health, Boxmeer, The Netherlands). “Intradermal” administration per se should not be equated with “needle-less” administration. Only when a needle-less device is “configured for intradermal vaccination”, then a vaccine may indeed be delivered (at least partly) into the dermis. Needleless intradermal administration is less invasive than needle injection and produces fewer adverse systemic effects within the animals and elicits an excellent immune response. It also reduces the risk of needles transmitting disease between pigs during the vaccination process.

However, the provision of a safe and efficacious vaccine suitable for intradermal administration is difficult, as the volume of a vaccine needs to be very small, typically in the range of about 0.1 to 0.5 ml. Therefore, the immunogens as well as the other components of the vaccine, such as possible adjuvants, need to be very concentrated, which increases the risk of interactions between the various vaccine components. As mentioned above, vaccines combining immunogens from PCV2 and Mhyo are commercially available. However, these combination vaccines are for intramuscular administration, and are not suitable for intradermal administration.

### OBJECT OF THE INVENTION

It was an object to provide a combination vaccine against diseases associated with infections by PCV2 and Mhyo suitable for intradermal administration. In particular, it was an object to provide a safe and efficacious vaccine that can be used for prophylactic treatment of an animal against infections by PCV2 and Mhyo.

### SUMMARY OF THE INVENTION

Surprisingly, it was found that these objects can be met, and consequently one or more of the disadvantages of the prior art can be overcome, by devising a particular adjuvant that enables a combination vaccine comprising non-replicating immunogen of porcine circo virus type 2 (PCV2) and non-replicating immunogen of Mycoplasma hyopneumoniae (Mhyo), wherein the combination vaccine is an oil-in-water emulsion comprising squalane, vitamin E-acetate and silica.

In particular, it was surprisingly found that a combination vaccine comprising non-replicating immunogen of PCV2 and non-replicating immunogen of Mhyo, wherein the combination vaccine is an oil-in-water emulsion (i.e. an emulsion of which the continuous phase is aqueous, having dispersed therein a hydrophobic liquid, the discontinuous phase, which latter phase may on itself have a secondary or further phase dispersed therein) comprising squalane, vitamin E-acetate and silica, can be used in the safe prophylactic treatment of an animal against an infection with PCV2 and an infection with Myho by intradermal administration.

The invention is also embodied in the adjuvant composition itself, in particular an adjuvant composition for formulating a non-live vaccine, wherein the composition is an oil-in-water emulsion comprising squalane, vitamin E-acetate and silica.

### Definitions

A “combination vaccine” is a vaccine comprising immunogens from more than a single species of micro-organism. A combination vaccine according to the invention at least comprises immunogens from porcine circo virus type 2 and from Mycoplasma hyopneumoniae. A combination vaccine according to the invention may thus colloquially also be referred to as a vaccine ‘against’ PCV2 and Mhyo.

A “vaccine” is commonly referred to as a pharmaceutical composition that is safe to administer to a subject animal, such as swine, and is able to induce protective immunity in that animal against a pathogenic micro-organism. A vaccine typically comprises an immunologically active component, and a pharmaceutically acceptable carrier. The ‘immunologically active component’, is one or more immunogenic molecule(s), such as non-replicating immunogens from PCV2 and Mhyo. These are recognised by the immune system of a target animal and induce a protective immunological response.

A vaccine generally is efficacious in reducing the severity of an infection, for example by reducing the number of the pathogens, or shortening the duration of the pathogen's replication in a host animal. Also, or possibly as a result thereof, a vaccine generally is effective in reducing or ameliorating the (clinical) symptoms of disease that may be caused by such infection or replication, or by the animal's response to that infection or replication.

“Non-replicating immunogen” of a pathogen is any substance or compound corresponding to the pathogen, other than the live replicating pathogen as a whole (either in wild type of attenuated form), against which pathogen an immunological response is to be elicited, such that the corresponding virulent pathogen or one or more of its virulence factors will be recognized by the host's immune system as a result of this immune response and are ultimately at least partly neutralized. Typical examples of non-replicating immunogens are killed whole pathogens (which term includes these pathogens in lysed form) and subunits of these pathogens such as capsid proteins, surface expressed molecules (for example recombinantly expressed proteins or lipopolysaccharides) and excreted molecules such as toxins.

“Prophylactic treatment” against an infection with a pathogen is typically aiding in preventing, ameliorating or curing an infection with that pathogen or a disorder arising from that infection, resulting from a post treatment challenge with the pathogenic pathogen, in particular to reduce its load in the host after such challenge and optionally to aid in preventing or ameliorating one or more clinical manifestations resulting from the post treatment infection with the pathogen.

### EMBODIMENTS OF THE INVENTION

In a first embodiment, the present invention relates to a combination vaccine comprising non-replicating immunogen of PCV2 and non-replicating immunogen of Mhyo, characterized in that the combination vaccine is an oil-in-water emulsion comprising squalane, vitamin E-acetate and silica.

Each of the “non-replicating immunogens” in the combination vaccine according to the invention can be of a single type, or can be of multiple types, e.g. from one or from more than one strains of the respective pathogen. For the invention, the non-replicative immunogen of PCV2 is preferably an inactivated whole PCV2 virus. Even more preferred is the use of the ORF2 protein as a subunit, which is typically obtained from a recombinant expression system, or is delivered and expressed via a replicon particle. PCV2 ORF2 can be expressed by a recombinant baculovirus in an insect cell culture and harvested. A replicon particle is a defective virus particle, for example an alphavirus particle as developed by AlphaVax. The parental PCV2 of the ORF2 sequence that is expressed can be of any of the PCV2 seroytypes a, b, c, or d, or can be from a chimera from one or more of these serotypes.

The non-replicating immunogen of Mycoplasma hyopneumoniae typically comprises killed whole Mycoplasma hyopneumoniae, i.e. killed Mhyo bacterin. The Mhyo bacterin is preferably from strain 11 or strain J.

An “oil-in-water emulsion” is an emulsion of which the continuous phase is aqueous, having dispersed therein a hydrophobic liquid, the discontinuous phase, which latter phase may on itself have a secondary or further phase dispersed therein. By the selection of the appropriate kind and concentration of emulsifier(s), such an emulsion can be formed. Procedures and equipment for the preparation of an oil-in-water emulsion for use as a vaccine are well-known in the art and are for instance described in handbooks such as: “Remington: the science and practice of pharmacy” (2000, Lippincot, USA, ISBN: 683306472), and: “Veterinary vaccinology” (P. Pastoret et al. ed., 1997, Elsevier, Amsterdam, ISBN 0444819681).

In the invention, the outer aqueous phase may comprise the non-replicating immunogens from PCV2 and Myho and the silica; and the oily phase may comprise squalane and vitamin E-acetate.

The combination vaccine according to the invention was found to be very effective, safe and stable, when prepared as an oil-in-water emulsion. Embodiments and preferences for the manufacture of an oil-in-water emulsion for the combination vaccine according to the invention will be described herein below.

“Squalane” is a non-mineral oil and also referred to as hydrogenated shark liver oil, hexamethyltetracosane, or perhydrosqualene. This is not to be confused with squalene (CAS nr. 111-02-4) which is a poly-unsaturated C30 oil and is metaboliseable as a compound of the cholesterol pathway. However, squalane is the fully hydrogenated form of squalene and is therefore not prone to oxidation. Thus, while squalane may be transported from the injection site (it thus “disappears” from the injection site) and therefore sometimes is indicated as being “metabolisable”, it is in fact an inert, non-metabolisable oil (it is only physically transported away from the injection site and not metabolised).

Originally the precursor to squalane was obtained from shark livers, but over environmental concerns this has shifted to other natural sources, such as olive oil, or to chemical synthesis. Therefore, included in the definition of squalane are natural, synthetic or semi-synthetic forms, or mixtures thereof.

Squalane is commercially available in a variety of purities, for example: from vegetable source, from Worlee (Squalane, vegetable), or Croda (Pripure Squalane); or synthetic, e.g. from Kuraray (Squalane-PE). For the invention, a high purity of the squalane is preferred: preferably over 75% purity, more preferably over 80, 90, or even over 95% purity, in that order of preference.

The squalane in the combination vaccine according to the invention is typically present in an amount of 1 to 15% w/v of the vaccine. More preferably, squalane is present in an amount of 3 to 12% w/v, or even 5 to 9% w/v of the vaccine, in that order of preference, for example at 5%, 6%, 7%, 8% or 9% w/v. Most preferred: squalane is present in an amount of about 6.8% w/v of the vaccine.

Therefore, in an embodiment of the combination vaccine according to the invention, the vaccine comprises squalane in an amount of 1 to 15% w/v.

“Vitamin E-acetate” is an acetate-ester of vitamin E (tocopherol) and can be derived from vegetable materials such as seeds, nuts, fruits or leaves, or from fatty meats, but may also be produced synthetically. Some alternate names are: tocopheryl acetate, or alpha-tocopherol-acetate. Included in the definition of vitamin E-acetate are natural, synthetic or semi-synthetic forms, or mixtures thereof. Vitamin E-acetate is commercially available, in different degrees of purity. The vitamin E-acetate for use in the combination vaccine according to the invention may be DL-alpha-tocopherol-acetate, which is the racemate of the chemical with CAS number: 7695-91-2.

The vitamin E-acetate in the combination vaccine according to the invention is typically present in an amount of 2 to 20% w/v of the vaccine. More preferably, vitamin E-acetate is present in an amount of 4 to 16% w/v, or even 6 to 10% w/v of the vaccine, in that order of preference, such as 6%, 7%, 8%, 9% or 10% w/v. Most preferred: vitamin E-acetate is present in an amount of about 8% w/v of the vaccine.

Therefore, in an embodiment of the combination vaccine according to the invention, the vaccine comprises vitamin E-acetate in an amount of 2 to 20% w/v.

“Silica” is silicon dioxide. Silica's are widely described for use in adjuvant compositions, and are commonly denoted as pharma grade silica. All pharma grade silica's have in common that they are colloidal silicon dioxide and have been used in the pharmaceutical industry for almost 50 years. Within this type of silica's, different surface areas, hydrophilic and hydrophobic (e.g. methylated), crystalline or amorphous (such as fumed silica), as well as different granulation ratios are available, all commonly used in adjuvant compostions. An example of type silica that is preferably used in adjuvant compositions is amorphous silica, (which can be hydrophilic or hydrophobic, but preferably hydrophilic when used in the present invention). A common type of amorphous silica is fumed silica, which is also known as pyrogenic silica because it is produced in a flame, consists of microscopic droplets of amorphous silica fused into branched, chainlike, three-dimensional secondary particles which then agglomerate into tertiary particles.

The silica for use in the combination vaccine of the invention may have a particle size of 100 to 700 m²/gram, more preferably 300 to 500 m²/gram, further more preferably 350 to 410 m²/gram, most preferably about 395±25 m²/gram. The surface area can be determined by methods known in the art, such as by calculation using a nitrogen adsorption method of Brunauer (Brunauer, S. et al., J. Am. Chem. Soc., 60, 309 (1938)).

Such products are commercially available for example under the tradename Aerosil® or Aeroperl® (under which tradenames many variants such as different surface area's, hydrophobic and hydrophilic, crystallin or amorphous are available). An example is Aerosil® 380 by Evonik Resource Efficiency GmbH, Germany, or as S5130 by Sigma-Aldrich, having a particle size of about 0.007 μm, a pH (4% dispersion) of 3.7 to 4.5, and a tapped density of about 50 g/l.

The silica in the combination vaccine according to the invention is typically present in an amount of 0.02 to 2% w/v of the vaccine. More preferably, silica is present in an amount of 0.05 to 1.0% w/v, or even 0.1 to 0.4% w/v of the vaccine, in that order of preference, such as 0.1%, 0.2%, 0.3% or 0.4% w/v. Most preferred: silica is present in an amount of about 0.2% w/v of the vaccine.

Therefore, in an embodiment of the combination vaccine according to the invention, the vaccine comprises silica in an amount of 0.02 to 2% w/v.

The combination vaccine of the invention typically contains a pharmaceutically acceptable carrier, which preferably is water. Preferably the water is of a high degree of purity, such as double distilled-, micro-filtrated-, or reversed-osmosis water. More preferred: the water is water-for-injection, and is sterile and essentially free from pyrogens.

A convenient feature of vaccines based on oil-in-water emulsions is that the immunogens are usually in the water phase. This means that the oily phase can be prepared and emulsified in water separately, employing methods and techniques that would not as such be compatible with maintaining the quality or the viability of the vaccine immunogens; for example using high-energy emulsification at high temperatures. This generates an oily emulsion for the invention, which is an oil-in-water emulsion of squalane, vitamin E-acetate and silica in water. To prepare the combination vaccine according to the invention, the aqueous phase with the immunogens and the silica, and the oily emulsion with the other adjuvants are combined by gentle mixing at room temperature.

The combination of the two compositions causes a dilution of each of them. Consequently, each needs to be prepared as an intermediary composition in which the concentration of the various components is higher than it will be in the final vaccine, by a factor equal to the dilution that will be applied. Typically, aqueous phase and oily emulsion can be mixed in a volume ratio anywhere between 10:90 and 90:10.

The combination vaccine according to the invention preferably comprises an aqueous phase and an oily emulsion—both as described—, in a volume ratio between 20:80 and 80:20. Therefore, in an embodiment, the combination vaccine according to the invention is prepared from the admixture of an aqueous phase and an oily emulsion, in a volume ratio between 20:80 and 80:20. Preferably the volume ratio is between 30:70 and 70: 30; between 40:60 and 60:40; or even the volume ratio is about 50:50, in that order of preference.

Evidently, when the combination ratio of aqueous phase and oily emulsion is about 50:50, then each of the two compositions should comprise its various components in an amount or in a concentration that is two times higher than desired in the final vaccine formulation that is prepared from the combination of the two intermediary compositions.

In a preferred embodiment, the oily emulsion for the invention is prepared using an emulsifier with an HLB value (hydrophilic-lipophilic balance) of 8 to 20; a preferred emulsifier is polysorbate 80. Polysorbate 80 refers to a chemical with CAS nr. 9005-65-6, also named: polyoxyethylene sorbitan monooleate. It has HLB value of approximately 15, and is widely commercially available, e.g. as Tween 80.

Preferably, polysorbate 80 is present in the combination vaccine according to the invention in an amount of 0.5 to 10% w/v of the vaccine. More preferably, polysorbate 80 is present in an amount of 0.7 to 7% w/v, 1.0 to 5%, or even 2 to 4% w/v of the vaccine, in that order of preference. Most preferred: polysorbate 80 is present in an amount of about 3.2% w/v of the vaccine.

Therefore, in an embodiment, the combination vaccine according to the invention comprises polysorbate 80 in an amount of 0.5 to 10% w/v.

An oily emulsion for the invention, can be prepared at any scale and using any suitable homogenisation equipment, such as from: Microfluidizer™, Silverson™, Ultra Turrax™, or a Dispax reactor (IKA). The skilled person can perform and optimise such an emulsification process to control the size of the particles of the dispersed phase (here: the oily adjuvants). Together with the choice of type- and concentration of the emulsifier(s), this controls the pharmaceutical properties of the emulsion, and also its stability. The main parameters of the emulsification process itself are: the energy input (power and rpm), the temperature, the duration, and the number of repeat cycles. Details of embodiments of the emulsification process are presented below.

The size of the particles of the dispersed phase is preferably quite small. When the diameter of the particles of the dispersed phase is below about 1 micrometre, such emulsions are commonly called “submicron emulsions”. In an embodiment of the oil-in-water emulsion of the combination vaccine according to the invention, the emulsion is a submicron emulsion.

Equipment to measure particle sizes of 1 micrometre or less is generally available, for example by laser diffraction measurement. Typically, particle size is expressed in nanometres (nm), and as an average particle size, also known as median diameter, expressed as the D50 of a cumulative particle size distribution.

For the invention, the particle size is expressed in nm of D50, as determined using a Mastersizer® (Malvern Instruments). Particle size measurements can be made in the (concentrated) oily emulsion or in the combination vaccine; the particle refractive index of the oily phase for the invention is 1.48. The Malvern Mastersizer size analysis report presents D50 as D(0.50). Therefore, in an embodiment of the submicron oil-in-water emulsion of the combination vaccine according to the invention, the oil-droplets have a D50 of 500 nm or less; preferably D50 is 250 nm or less. More preferred: D50 is 150 nm or less.

There are many ways available to produce such submicron emulsions, typically by the use of a high-energy emulsification process, for example using: high-pressure homogenisers, rotor-stator devices, blenders, ultrasonic waves, microporous membranes, or microchannelling devices.

A preferred process for high-energy emulsification for the invention is the use of a high-pressure homogeniser, preferably a Microfluidizer™ (Microfluidics). Typically, 3 passages at a pressure of 500 to 1500 bar (i.e. 7000 to 22000 psi) will be sufficient. Emulsions prepared in this way typically have dispersed phase particles with a D50 of 500 nm or less, and have a narrow size distribution; for the invention, the dispersed phase are the droplets of the oily adjuvants.

Typically, emulsions with such very finely sized particles of the dispersed phase, are prepared in several steps. In this way, an initial relatively coarse oily emulsion is prepared by low-energy mixing, which is followed by one or more subsequent high-energy treatments to achieve further reduction of particle size. Next, the ‘microfluidised’ oily emulsion, comprising the adjuvants and optionally the emulsifier in water is then combined with the aqueous phase comprising the immunogens, to prepare the combination vaccine according to the invention.

For reasons of product consistency and quality, not only the median particle diameter, but also the spread in particle size, also known as the size distribution, can advantageously be monitored and controlled. The size distribution of the oil-droplets in the submicron oil-in-water emulsion of the combination vaccine according to the invention is preferably relatively narrow. An indicator of particle size distribution is the D90 of a cumulative particle size distribution.

Therefore, in an embodiment of the submicron oil-in-water emulsion of the combination vaccine according to the invention, the oil-droplets have a D90 below 900 nm, more preferred D90 is below 500 nm, 400 nm, or even below 300 nm, in that order of preference. Most preferred: D90 is about 150 to 250 nm.

One of the advantages of the emulsion having such small particle size, and distribution, is that this can then be sterilised by filtration, without significant loss of material, because typical sterilisation filters have a pore size of about 0.2 micrometres. Such filter sterilisation overcomes the need for other methods of sterilisation that may be damaging to the quality of the components of the oily emulsion, such as by: heating, chemicals, or irradiation.

A combination vaccine according to the invention therefore typically comprises the non-replicative immunogens from PCV2 and Mhyo in amounts that are capable of inducing in the animal target a protective immune response against their associated diseases, as described above.

A skilled person in the field of the invention will be more than capable of determining the effectiveness of a combination vaccine according to the invention, e.g. by monitoring the immunological response following vaccination or after a challenge infection, e.g. by monitoring the targets' signs of disease, clinical scores, or by re-isolation of the pathogen, and comparing these results to a vaccination-challenge response seen in mock-vaccinated animals.

As an indication, the amounts of the immunogens to be used in the combination vaccine according to the invention can be based on those used in the respective monovalent- or combination vaccines with these immunogens. For example, the combination vaccine according to the invention can comprise per millilitre: 1 to 150 μg ORF2 of PCV2; and Mhyo: 2-50% w/v of an inactivated concentrated Mhyo culture. Methods to quantify these immunogens are well-known in the art, and can also rely on ELISA based quantification against specific standards.

The combination vaccine according to the invention can advantageously be combined with one or more further antigens or immunogens, replicative or non-replicative, whole or disrupted. Therefore, in an embodiment the combination vaccine according to the invention may comprise at least one additional antigen or immunogen.

The additional antigen or immunogen is either an attenuated form of a micro-organism which is pathogenic to swine, or is a non-replicative antigen or immunogen derived from a micro-organism pathogenic to swine. The micro-organism may be any virus, bacterium, parasite, fungus, rickettsia, protozoa and/or parasite that is pathogenic to swine. Examples of such micro-organisms pathogenic to swine are: pseudorabies virus, porcine parvo virus, classical swine fever virus, swine influenza virus, foot-and-mouth disease virus, porcine epidemic diarrhoea virus, transmissible gastro enteritis virus, porcine respiratory coronavirus, vesicular stomatitis virus, Lawsonia intracellularis, Actinobacillus pleuropneumoniae, Brachyspira, E. coli, Haemophilus, Streptococcus, Salmonella, Clostridia, Pasteurella, Erysipelothrix, Leptospira, Bordetella, Toxoplasma, Isospora, and Trichinella. Preferred additional antigens or immunogens are one or more from: Lawsonia intracellularis, Actinobacillus pleuropneumoniae, Haemophilus parasuis, Brachyspira hyodysenteriae, and swine influenza virus.

Observed effects of the combination vaccine according to the invention are:

for Mhyo: prevention or reduction of lung lesions caused by Mhyo, such as consolidated pneumonia, and chronic respiratory disease. For Mhyo, the most reliable measure of vaccine potency is the reduction of lung lesion scores after Mhyo challenge infection. Such lesions are typically scored during necropsy by macroscopic assessment of lung consolidation, based on the Goodwin scale (Goodwin et al., 1969, J. Hyg. Camb., vol. 67, p. 465-476); this scale runs from zero up to a maximum of 55 points/animal for totally affected lungs.

for PCV2: prevention or reduction of clinical signs of wasting or ill thrift, presence of gross and microscopic lesions characteristic of the disease, and presence of viral antigen or DNA in the microscopic lymphoid lesions. For PCV2, the most reliable measure of vaccine potency is the testing of the presence of viral nucleic acid by qPCR in serum, faecal swab material, nasal swab material, inguinal lymph node, mesenteric lymph node, tonsil and lung. The induction of antibodies against PCV2 after vaccination correlates with protection.

In a preferred embodiment, the combination vaccine is for swine. The term “swine” refers to animals of the family Suidae, and preferably to animals of the genus Sus, which are also referred to as porcines. Examples are: a wild or a domestic pig, hog, wild boar, babirusa, or warthog. This also includes swine indicated by an arbitrary name, for example referring to their sex or age such as: sow, queen, boar, barrow, hog, gilt, weaner, or piglet. Further the term swine refers to porcine animals of any type such as of breeding- or fattening type, and to parental lines of any of these types.

The combination vaccine according to the invention can be composed in different ways, as described herein.

In one embodiment, the combination vaccine according to the invention is provided as a ready-to-use formulation, i.e. as a formulation in which all components of the vaccine are readily mixed so that the combination vaccine can be directly used for vaccination without the necessity of any further mixing or reconstitution steps.

In an alternative embodiment, the combination vaccine according to the invention can be produced from a kit of parts, comprising at least two containers: one container comprising all components of the combination vaccine according to the invention except for the Myho immunogen; and one container comprising the Myho immunogen. The PCV or Myho immunogen can be provided, for example, in freeze-dried form, or as a sterile suspension, such as an aqueous suspension. The freeze-dried form can be a freeze-dried cake in a container, e.g. a bottle, but can also be a lyosphere as applied in the Sphereon™ technology.

The elements of the kit of parts then together embody the combination vaccine according to the invention. The contents of the at least two containers can be reconstituted in situ directly before use, i.e, prior to vaccination. Upon the reconstitution of the non-replicating immunogen from Mhyo the complete combination vaccine according to the invention is formed. This is also referred to as mixing the vaccine ‘on-the-spot’, or ‘field-side’ mixing.

Therefore, in a further aspect, the invention relates to a kit of parts comprising at least two containers: one container comprising non-replicating or immunogen from PCV2 in an oil-in-water emulsion comprising squalane, vitamin E-acetate and silica; and one container comprising non-replicating immunogen from Mhyo.

In an alternative embodiment both immunogens of PCV2 and Mhyo can be provided in one container, for example as aqueous solution or dispersion or in lyophilized form, optionally further containing the silica, and which is reconstituted before use with the components of a second container containing the (other) adjuvants in the form of an o/w emulsion.

The kit of parts according to the invention, and its elements, can comprise any of the embodiments (preferred or not) as described herein for the combination vaccine according to the invention, or any combination of two or more of those embodiments of the combination vaccine according to the invention.

Therefore, in a further aspect the invention relates to a method for the preparation of a combination vaccine according to the invention, comprising the steps of:
preparing an aqueous phase comprising non-replicating immunogen from PCV2 and Mhyo, and
admixing said aqueous phase with an oily emulsion comprising squalane, vitamin E-acetate and silica in order to form an oil-in-water emulsion.

In an alternative aspect, the invention relates to a method for the preparation of a combination vaccine according to the invention, comprising the steps of:
preparing an aqueous phase comprising non-replicating immunogen from PCV2,
admixing said aqueous phase with an oily emulsion comprising squalane, vitamin E-acetate and silica in order to form an oil-in-water emulsion, and
admixing said oil-in-water emulsion with non-replicating immunogen from Mhyo.

In a further aspect, the invention relates to a method for the preparation of a combination vaccine according to the invention, comprising the steps of:
preparing non-replicating immunogen from Mhyo in a freeze-dried form,
preparing an aqueous phase comprising non-replicating immunogen from PCV2,
admixing said aqueous phase with an oily emulsion comprising squalene, vitamin E-acetate and silica, and
reconstituting said freeze-dried non-replicating immunogen from Mhyo with said admixture of aqueous phase and oily emulsion.

In yet a further aspect, the invention relates to a method for the preparation of a combination vaccine according to the invention, comprising the steps of:
preparing non-replicating immunogen from PCV in a freeze-dried form,
preparing an aqueous phase comprising non-replicating immunogen from Mhyo and silica,
admixing said aqueous phase with an oily emulsion comprising squalane and vitamin E-acetate, and
reconstituting said freeze-dried non-replicating immunogen from PCV with said admixture of aqueous phase and oily emulsion.

At different points in these methods, additional steps may be added, for example for additional treatments such as for purification or storage. Also the method for the preparation can involve the admixing with an additional antigen or immunogen, or pharmaceutically acceptable excipients such as stabilisers or preservatives.

As described, the combination vaccine according to the invention, which can be prepared by a method according to the invention, can advantageously be used for the intradermal administration to swine, to protect against infection by and/or disease associated with an infection by PCV2 and Mhyo.

Therefore, in a further aspect the invention relates to an oil-in-water emulsion comprising squalane, vitamin E-acetate, silica, non-replicative immunogens from PCV2 and Mhyo, for use in vaccination into the dermis of an animal, such as swine, against PCV2 and Mhyo.

In a further aspect the invention relates to the use of non-replicative immunogens from PCV2 and Mhyo, for the manufacture of a combination vaccine for an animal, such as swine, characterised in that the vaccine is an oil-in-water emulsion comprising squalane, vitamin E-acetate and silica.

The combination vaccine according to the invention can be applied for the vaccination of swine against PCV2 and Mhyo.

Therefore, in a further aspect the invention relates to a method for the vaccination of an animal, such as swine, against PCV2 and Mhyo, by intradermal administration to said animal of an oil-in-water emulsion comprising squalane, vitamin E-acetate, silica, and non-replicative immunogen from PCV2 and Mhyo.

Or in a similar embodiment: the invention relates to a method for the vaccination of an animal, such as swine against PCV2 and Mhyo, by intradermal administration to said animal of a combination vaccine according to the invention.

Therefore, the combination vaccine according to the invention is typically administered into the skin of the animal, i.e. is applied by intradermal administration. This can be achieved in different ways, for example using a classic syringe and hypodermic needle. Alternatively, the parenteral administration may be done by some method of needle-free injection, delivering the vaccine by an intradermal applicator, such as the IDAL® applicator from MSD Animal Health.

In order to be suitable for intradermal administration, the volume of an animal dose of the combination vaccine according to the invention is typically 0.05 to 1.0 ml per animal; preferably 0.1 to 0.5 ml, more preferably about 0.2, 0.3 or 0.4 ml, most preferably about 0.2 ml per animal dose, in that order of preference.

The administration regime for a method of vaccination according to the invention to a target swine can be in single or in multiple doses, or in a manner compatible with practical aspects of swine husbandry.

When required, the animal target may be given second or further administrations of the combination vaccine according to the invention, later in life, so-called booster vaccinations. However, the combination vaccine according to the invention is optimised in such a way that a single vaccination dose will generally suffice to provide an immune protection during the relevant period of life of the animal, for example during the fattening stage of a swine up to 6 months of age.

Thus, in a preferred embodiment, the combination vaccine according to the invention is administered only once per animal target, i.e. it is a single-dose vaccine.

Preferably, the regime for the method of vaccination is integrated into existing vaccination schedules of other vaccines that the target swine may require, in order to further reduce stress to the animals and to reduce labour costs. These other vaccines can be administered in a simultaneous, concurrent, or sequential fashion, in a manner compatible with their registered use. Therefore, in an embodiment of the method of vaccination of swine according to the invention, the combination vaccine according to the invention is administered in a combination with another swine vaccine.

In case the target animal is swine, a target swine for a vaccination for the invention can be of any age in which they are susceptible to the vaccination, and/or are susceptible to the disease or the infection against which the vaccine protects. Therefore, in an embodiment of the method of vaccination of swine according to the invention, the combination vaccine according to the invention is administered to young swine, i.e. swine of about 2 months of age. Alternatively, the combination vaccine according to the invention is administered to adult swine, i.e. swine from about 6 months of age.

Because of the high prevalence of Mhyo and PCV2 and because of the widespread use of vaccines against one or more of these pathogens, many swine sows will be seropositive for antibodies against one or more of Mhyo and PCV2. Consequently, young swine that consumed colostrum from such sows, can be be MDA+(maternally derived antibody positive). This is no hindrance to the efficacy of the combination vaccine according to the invention, as it is effective also in MDA+swine. Therefore, in an embodiment of the method for vaccination according to the invention, the combination vaccine according to the invention is administered to MDA+swine.

The administration of a combination vaccine according to the invention can be applied either as a prophylactic- or as a therapeutic treatment, or both, as it interferes both with the establishment and with the progression of an infection by Mhyo and PCV2. The use of the combination vaccine according to the invention will assist in the reduction of infection by one or both of Mhyo and PCV2 in a swine herd, on a farm, or in swine in a geographical area. Therefore, in a further aspect the invention relates to a method for the reduction of an infection with Mhyo and PCV2, or of associated signs of disease in swine, characterised in that the method comprises the intradermal administration to said swine of a combination vaccine according to the invention.

The invention will now be further described by the following, non-limiting, examples.

### EXAMPLES

### Example 1: Preparation of the Combination Vaccine

The combination vaccine according to the invention was prepared as follows:

This oily emulsion in 2× concentration was prepared according to the following subsequent process steps:
required amounts of vitamin E-acetate and squalane were weighed off, and combined in a beaker,
the vitamin E-acetate/squalane mixture was homogenised by low-energy mixing (magnetic stirrer), at room temperature,
the required amount of Polysorbate 80 was weighed off, and added to the homogenised vitamin E-acetate/squalane mixture,
the combined mixture was homogenised again, by low-energy mixing at room temperature,
the homogenised mixture was sterilised by filtration through an 0.2 micrometre filter (Pall, Ultipor™ N66),
the required amount of (heat sterilised) silica was weighed off and added to the homogenised mixture, after which the combined mixture was homogenised again, by low-energy mixing at room temperature,
the mixture was heated to 65-75° C.,
the water for injection (sterilised) was heated to 65-75° C.,
the heated oil-phase and the water were pre-mixed using high energy mixing by Ultra Turrax with N18 rod, for 5-15 minutes; the temperature decreased from 65 to 55° C.
the pre-mix was given 3 passages through a Microfluidizer™ at 800 bar; temperature was kept below 50° C. with a cooling spiral.

Of the final oily emulsion, completeness and level of homogenisation were checked by light-microscopy. Further pH (7.34), and osmolality (221 mOsm/kg) were also checked. Particle size measurements revealed: D100=300 nm; D99=250 nm; D90=200 nm, and D50=130 nm.

The aqueous phase (in 2× concentration) was prepared by taking the required amount of each of the non-replicating immunogens: Mhyo: 6% v/v of a 10× concentrated inactivated culture and PCV: 50 μg ORF2.

Next, both concentrated compositions (oily emulsion with adjuvants, and aqueous phase with immunogens) were combined in an approximate 50:50 volume ratio, by low-energy mixing at room temperature.

The following vaccine adjuvant compositions are prepared as oil-in-water (o/w) emulsions (all percentages are % w/v) using the procedure described above. To some compositions aluminum hydroxide was added in the specified amount (as double concentrate) together with the Polysorbate 80 and squalane when preparing the oily emulsion:

TABLE 1

| | | | Reference | Reference |
|---|---|---|---|---|
| | Composition | Composition | Composition | Composition |
| | 1 | 2 | 3 | 4 |
| Polysorbate 80 | 3.24% | 3.24% | 3.24% | 3.24% |
| (d = 1.08 g/ml) | | | | |
| Squalane | 6.75% | 6.75% | 6.75% | 6.75% |
| (d = 0.81 g/ml) | | | | |
| Vitamin | 7.94% | 7.94% | 7.94% | 7.94% |
| E-acetate | | | | |
| (d = 0.95 g/ml) | | | | |
| Aerosil 380 | 0.2% | 0.2% | — | — |
| Water for | 28.78% | 28.78% | 31.97% | 28.98% |
| injection | | | | |
| Alhydrogel | — | 9.83% | 1.73% | 9.83% |
| (Al(OH)₃ 3%) | | | | |
| PBS** + | Balance | Balance | Balance | Balance |
| Immunogen | | | | |

*Phosphate Buffered Saline

### Example 2: Efficacy of PCV2/Mhyo ID Formulations in Pigs Against Mhyo Challenge Infection

The effectiveness of vaccination with Mhyo intradermal (ID) formulations (0.2 ml, vaccinated at the right side of the neck with the IDAL® vaccine) against Mhyo challenge is tested in Specific Pathogen-Free (SPF) piglets (ToJaPigs). The formulations have been prepared with various treatments of Mhyo immunogen and are formulated at 5 PCVU/ml (approx. 25% w/v of an inactivated Mhyo culture). Animals were vaccinated at 3 weeks of age according to the following scheme (Table 2). Four weeks after vaccination all animals were infected with Mhyo. All animals were challenged on two consecutive days at 7 weeks of age, i.e. 4 weeks post vaccination, with 10 ml intra-tracheal Mhyo strain 98 with 10⁹ and 10⁹ CCU/ml respectively. Three weeks post challenge, the animals were sacrificed and the extent of Mhyo-induced consolidated pneumonia was scored according to Goodwin (maximum score: 55).

TABLE 2

| Group | Immunogens | | |
|---|---|---|---|
| (N) | PCV2 | Mhyo | Adjuvant |
| 1 (15) | X | X | Reference Composition 3 |
| 2 (15) | X | X | X-Solve12* + Aerosil380 |
| 3 (15) | — | — | Non-vaccinated control group |

*Proprietary adjuvant of Porcilis ^{®} PCV ID (MSD Animal health)

Results:

Effectiveness of vaccination is determined by lung lesion scores (LLS, averaged), which were recorded for each pig and compared to the non-vaccinated control group.

TABLE 3

| Group | Lung Lesion Score (LLS) |
|---|---|
| 1 | 13.2 |
| 2 | 11.5 |
| 3 | 15.4 |

No significant reduction in lung lesion score was obtained with any of the vaccines tested in groups 1 and 2 compared to the control group. Instead, lung lesion core was similar as in the non-vaccinated control group.

Thus, neither the combination of the adjuvants allohydrogel, squalane, vitamin E acetate and silica in combination with the immunogens of PCV2 and Mhyo nor the combination of the commercially available PCV vaccine Porcilis® PCV ID and silica as adjuvant was suitable for safe and effective intradermal administration.

### Example 3: Efficacy of a PRRS Vaccine Reconstitituted in PCV2/Mhyo ID Formulations in Pigs

The objective of this study was to compare the safety and serological efficacy of different PCV2 and/or Mhyo vaccines containing Aerosil200 or Aerosil380 reconstituted with Porcilis® PRRS, when administered intradermally (ID) in the neck of 5 weeks-old piglets. Piglets were allotted to the treatment groups indicated below (Table 4). The piglets were vaccinated intradermally when they were approximately five weeks old. Piglets from groups 4 and 5 were vaccinated with Porcillis PRRS with 10^{4.5} TCID₅₀ virus reconstituted in different PCV2-Mhyo vaccine formulations as described below

TABLE 4

| Group | Immunogens | | |
|---|---|---|---|
| (N) | PCV2 | Mhyo | Other components |
| 4 (10) | X | X | Porcilis ^{®} PRRS + Aerosil200 |
| 5 (10) | X | X | Porcilis ^{®} PRRS + Aerosil380 |
| 6 (10) | X | — | Porcilis ^{®} PCV ID + Porcilis ^{®} |
| | | | PRRS; non-mixed (positive control) |
| 7 (10) | — | — | Non-vaccinated control group |

Piglets from group 4 were vaccinated intradermally with a single dose (0.2 ml) of vaccine formulated with PCV2 (10000 AU/ml; approx. 80 μg/ml;)+M. Hyo (10 PCVU/ml) and the adjuvant Aerosil200.
Piglets from group 5 were vaccinated intradermally with a single dose (0.2 ml) of vaccine formulated with PCV2 (10000 AU/ml)+M. Hyo (10 PCVU/ml) and the adjuvant Aerosil380.
Piglets from group 6 were vaccinated intradermally with a single dose (0.2 ml each) of vaccine Porcilis® PCV ID+Porcilis® PRRS, non-mixed.
Piglets from group 7 were not vaccinated (negative control group). All animals were examined for injection site reactions.

All piglets were observed daily after vaccination for clinical signs. Temperatures were taken and serum samples were collected from all animals. Samples were tested for antibodies against PCV2, M. hyopneumoniae and PRRSV.

Results:

Following vaccination, the body temperatures of all groups were comparable. At the day of vaccination all animals had comparable PCV2 antibody titers. PCV2 antibody titers remained at a constant level in all groups until the end of the study. The antibody titers of the control group 7 decreased over time. At the day of vaccination all animals were negative for PRRS antibodies. Following all groups showed 0-20% PRRS responders. The control group 7 remained negative. At the start of the study all animals were serologically negative for M. hyopneumoniae. All groups showed 0-20% responders at SD21. At SD28 most groups showed positive animals. The control group 7 showed no M. hyopneumoniae positive response. Results of IgM antibody response (predictive for vaccine take) against PCV2 and Mhyo are shown in Tables 5 and 6.

TABLE 5
Percentage of PCV2 specific IgM positive
animals following vaccination

| Group | SD0 | SD14 | SD21 |
|---|---|---|---|
| 4 | 0 | 80 | 40 |
| 5 | 20 | 50 | 80 |
| 6 | 0 | 100 | 100 |
| 7 | 20 | 20 | 20 |

TABLE 6
Percentage of Mhyo serological antibody response
positive animals following vaccination

| Group | SD0 | SD14 | SD21 | SD28 |
|---|---|---|---|---|
| 4 | 0 | 0 | 20 | 20 |
| 5 | 0 | 0 | 0 | 10 |
| 6 | 0 | 0 | 0 | 10 |
| 7 | 0 | 0 | 0 | 0 |

From this study, it can be concluded that no vaccinated group showed an acceptable antibody response against PCV2 or Mhyo. Thus, neither the adjuvant Aerosil A380 nor Aerosil A200 when reconstituted with Porcilis® PRRS and PCV2 and Mhyo immunogen was suitable for safe and effective intradermal administration in pigs.

### Example 4: Efficacy of a PRRS Vaccine Reconstituted in Mhyo ID Formulations in Pigs Against Mhyo Challenge Infection

Effectiveness of vaccination with Mhyo intradermal (ID) formulations mixed with PRRS (A212D, 10^{5.1} TCID50/dose) against Mhyo challenge infection is tested in SPF piglets. The formulations have been prepared with various adjuvants. Animals were vaccinated ID (0.2 ml) in the right side of the neck at 3 weeks of age according to the following scheme (Table 7). Three weeks after vaccination all animals were infected with Mhyo.

TABLE 7

| Group | Immunogens | | |
|---|---|---|---|
| (N) | PRRS | Mhyo | Vaccine adjuvant |
| 8 (12) | X | X | Reference Composition 3 |
| 9 (12) | — | X | X-Solve (Porcilis ^{®} MHyo ID once), |
| | | | positive control for Mhyo |
| 10 (12) | X | — | Porcilis ^{®} PRRS, positive control for PRRS |

All animals were infected on two consecutive days at 6 weeks of age with 10 ml intra-tracheal Mhyo strain 98 with 9 and 8 CCU, respectively.

Results:

Effectiveness of vaccination is determined by lung lesion scores (LLS), which were recorded for each pig and compared to the non-vaccinated control group.

TABLE 8

| | Lung Lesion Score (LLS) | |
|---|---|---|
| Group | Score | % reduction |
| 8 | 9.0 | 22 |
| 9 | 3.0 | 74 |
| 10 | 11.5 | — |

It could be shown that the intradermal administration of a combination of Mhyo and PRRS immunogens with alhydrogel, squalane and vitamin E-acetate as adjuvant composition as in Reference Composition 3 gives a very low and thus unacceptable lung lesion score of 22% reduction versus 74% in the positive control group using a commercial Mhyo vaccine. Thus, the combination of the immunogens with the adjuvants alhydrogel, squalane and vitamin E-acetate was not suitable for safe and effective intradermal administration.

### Example 5: Efficacy of PCV2-Mhyo ID Formulations in SPF Pigs Against Mhyo Challenge Infection

Groups of 12 pigs were intradermally vaccinated according to the following scheme (Table 9) at the age of three weeks (+/−three days), group 17 was not vaccinated and serves as Mhyo challenge control. Four weeks after vaccination all animals were infected with a virulent Mhyo strain. Three weeks post-challenge all animals were post-mortem investigated for lung lesions. Prior to vaccination, before challenge and at post-mortem blood samples were taken.

TABLE 9

| Group | | Mhyo | PCV | Volume |
|---|---|---|---|---|
| (N) | Adjuvant/product | immunogen | immunogen | & Route |
| 11 (12) | Composition 1 | Strain 11 | PCV2 | 0.2 ml |
| 12 (12) | Composition 1 | Strain J | | ID, |
| 13 (12) | Composition 2 | Strain 11 | | neck |
| 14 (12) | Reference Composition 4 | Strain 11 | | right |
| 15 (12) | ISA28 + micro-fortasol* | Strain 11 | | side |
| | (Reference) | | | |
| 16 (12) | Porcilis ^{®} Mhyo | Strain 11 | — | |
| | ID Once** | | | |
| 17 (12) | —*** | — | — | — |

*Montanide ^{®} ISA 28 of Seppic to which microfluidized vitamin E acetate was added
**Positive control group for Mhyo
***Non-vaccinated control group

Experimental Procedures

Serology

Blood samples (vena jugularis) were taken just before vaccination (T=0, three weeks of age), just before challenge (T=4, 7 weeks of age) and at post-mortem (T=7, 10 weeks of age). Samples were transported at ambient temperature. From the blood samples serum was derived in duplicate. The presence of the relevant antibodies in all serum samples was determined in Elisa tests for M hyo and PCV according to standard procedures.

Clinical Observations and Rectal Temperature

Clinical observations were done and rectal temperatures were measured just before vaccination, four hours and one and two days after vaccination. Observations and temperature were noted.

Post Mortem Examination

At the end of this experiment, 4 weeks after challenge, the pigs were sedated by means of electrical stunning and subsequent euthanized by bleeding to death. Lung lesions were scored according to Goodwin.

Results:

No injection site reactions that were unacceptable were observed. No unacceptable temperatures were observed.

Effectiveness of vaccination is determined by lung lesion scores (LLS), which were recorded for each pig and compared to the non-vaccinated control group. Results are shown in Table 10:

TABLE 10

| | Lung Lesion Score (LLS) | |
|---|---|---|
| Group | Score | % reduction |
| 11 | 8.7 | 47% |
| 12 | 6.0 | 63% |
| 13 | 2.8 | 83% |
| 14 | 13.0 | 20% |
| 15 | 20.0 | −23% |
| 16 | 4.0 | 75% |
| 17 | 16.3 | — |

The above data of lung lesion scores show that groups 11, 12 (inventive compositions) and 13 (Reference Composition 2) gave acceptable scores, since the reduction compared to the non-vaccinated control group (group 17) was above 45% (which was used as cut-off value for substantial effectiveness). Instead, groups 14 and 15 gave unacceptable scores, as the LLS in group 15 was even higher than in the non-vaccinated control group, and the LLS in group 14 was only slightly reduced compared to the negative control group by 20%.

Thus, it could be shown that the inventive vaccine composition including the adjuvant combinations of squalane, vitamin E-acetate and silica gave acceptable lung lesion scores showing effective vaccination.

### Example 6: Efficacy of PCV2-Mhyo ID Formulations in SPF Pigs Against PCV Challenge Infection

Piglets were allotted to five treatment groups of 10 piglets each and vaccinated intradermally when they are approximately three weeks old. Piglets from group 1 through 3 were vaccinated with the vaccines described below. Group 4 was vaccinated with Porcilis® PCV ID and Porcilis® M Hyo ID ONCE as positive control group. Group 5 was not vaccinated (negative control). At three weeks post vaccination (6 weeks of age) all animals were challenged using 5.0 log10 TCID50/mL of wild-type PCV2b challenge virus strain 112/11 applied intranasally (3 ml per nostril). The treatment scheme is shown below in Table 11. Three weeks post challenge, all animals were necropsied and inguinal lymph node, mesenteric lymph node, tonsil and lung were sampled for the detection of PCV2.

All piglets were observed daily after vaccination for clinical signs. Serum samples were collected on the day of vaccination and on SD14, SD20, SD35 and SD42 (during necropsy). Samples were tested for antibodies against PCV2 viral nucleic acid by qPCR. Fecal swabs were collected on SD20, 35 and SD41 from all animals and were examined for PCV2 viral nucleic acid by qPCR.

TABLE 11

| Group | Adjuvant/ | Mhyo | PCV | Volume & |
|---|---|---|---|---|
| (N) | product | immunogen | immunogen | Route |
| 18 (10 | Composition 1 | Strain 11 | PCV2 | 0.2 ml ID, neck |
| 19 (10) | Composition 2 | | | right side |
| 20 (10) | ISA28* + | | | |
| | micro-fortasol | | | |
| 21 (10) | Porcilic ^{®} | | | 0.2 ml ID R1 |
| | PCV ID + | | | (PCV) + 0.2 |
| | Porcilis ^{®} | | | ml ID R2 (Mhyo) |
| | M Hyo ID | | | |
| | Once** | | | |
| 22 (10) | —*** | — | — | — |

*Montanide ^{®} ISA 28 of Seppic to which microfluidized mineral oil was added
**Positive control group for Myho and PCV2
***Non-vaccinated control group

Treatment

Dosage and Administration

Vaccinations were done by the intradermal route (0.2 ml), on the right side of the neck. Group 21 was vaccinated twice on the right side. Challenge was done by the intranasal route, 6 ml, 3 ml per nostril using a MAD applicator.

Test System

Only healthy animals were used. In order to exclude unhealthy animals, they were examined before vaccination (general physical appearance and absence of clinical abnormalities or disease). Just before vaccination, all animals will be individually numbered with eartags. All pigs were observed daily for clinical signs of disease. Observations will consist of systemic reactions such as loss of appetite, reluctance to move, tendency to lie down, listlessness or drowsiness, shivering, bristling, edema (especially around the eyes), vomiting and diarrhea and dyspnea.

Experimental Procedures

Sampling of Blood

Blood samples were collected from all animals on the day of vaccination, one day before challenge, two weeks later and at the day of necropsy. A minimum of 4 ml and a maximum of 8 ml blood was taken per animal. This was done from all pigs individually according to standard procedures. The blood samples were collected without the addition of anti-coagulant.

Fecal Swabs

Fecal swabs were taken one day before challenge infection, at 2wpc and one day before necropsy. Swabs were taken into medium containing antibiotics.

Post Mortem Examination

At 3 weeks post challenge the animals were transported to the necropsy room. They were anaesthetised using an electrocution apparatus and exsanguinated in accordance with standard procedures. The carcasses of the animals were destroyed according to standard procedures. During necropsy the animal was opened and the viscera are inspected in-situ, paying particular attention to the following organs: lungs, inguinal and mesenteric lymph nodes, tonsils, thymus, spleen, liver and kidneys. Subsequently, samples from tonsil, lung, mesenteric lymph node and inguinal lymph node were removed and divided in two parts, one part for freezing and analysis by PCV2 qPCR and one part for fixation followed by (immuno)histochemical analysis.

Processing of Samples

Serum was prepared from the clotted blood samples and aliquots (e.g. 2×0.8 ml) were filled. The samples were NOT heat-inactivated. Fecal samples were prepared from swabs and aliquots (e.g. 2×0.8 ml) were filled. Until use the samples were stored at ≤−15° C. The period between sampling and storage did not exceed 36 (serum) or 48 (swabs) hours.

PCV2 Antibody ELISA

Sera were tested for antibodies against PCV2 according to standard procedures. In brief, serially diluted serum samples were incubated on microtiter plates coated with baculovirus expressed PCV2 ORF2 antigen. After removing the sera, all wells were incubated with a fixed amount of biotin-labeled PCV2-specific monoclonal antibody (MoAb). Bound MoAb is then incubated with peroxidase-conjugated streptavidin followed by chromophoric detection. Titers were defined as the reciprocal of the interpolated serum dilution with an extinction value equal to the 50% maximum extinction for the test. Results were expressed as log₂ titers. Titers below 2.0 log₂ were considered negative. For negative samples, a value of 1.0 log₂ was used for calculation purposes.

Quantitative PCR

Quantitative PCR (qPCR), on PCV2 nucleic acid, was performed on all sera, fecal swabs, and on 10% tissue homogenates of tonsil, lung, mesenteric lymph nodes and inguinal lymph nodes according to standard procedures. In brief, DNA was extracted from the samples using a commercial kit. PCV2 genomic DNA in each sample was quantified by polymerase chain reaction (PCR), using primers and a probe specific for PCV2-ORF2. The cycle number where specific fluorescence exceeds the threshold is correlated with the cycle numbers for a set of samples containing known amounts of a PCV2-ORF2-containing plasmid. Results were expressed as log₁₀ copies/0 of extracted DNA (log₁₀ c/μl). Values lower than 1.00 log₁₀ c/μl are considered negative, were taken as 0.00 log₁₀ c/μl for calculation purposes.

Immunohistology (IHC)

Tonsil and lymph node samples were prepared for histological examination. Samples were fixed in 10% formalin, paraffin embedded, and immunohistochemistry was performed for detection of PCV2 antigen on slides. An anti-PCV2 rabbit serum was used as primary antibody and Envision+ (DAKO, Denmark) was used as detection system, according to the manufacturer's instructions. The slides were counterstained with hematoxylin. Microscopic examination was done. For tonsil and lymph nodes, characteristic brown staining was scored on an ordinal scale as follows:
0 No specific positive staining cells observed
1 scattered (single) positive staining cells present in (less than), 10% of follicles
2 positive staining (single) cells observed in 10-50% of follicles, or focal collections of >15 positive staining cells in <10% of the follicles.
3 specific staining in >50% of follicles

The results were recorded as the total score, being the sum of scores of the individual tissues.

Results:

Pcv2 Serology:

At start of measurement at SD1, log 2 titers of all groups was between 4 and 5. The positive control (group 21) gave a log 2 titer of 10 between SD34 and SD41. In the same week, the log 2 titers in the groups 18, 19 and 20 was between 8 and 10. Non-vaccinated negative control group 22 led to a log 2 titer below 4.

PCV2 Viral Load in Serum (qPCR Serum)

Between SD0 (start of measurement) and SD19, no viral load could be detected in all groups (log 10 c/μl=0).

At SD 34 viral load in the positive control (group 21) increased to about 1.0 log 10 c/μl and further increased to about 1.6 at SD41.

At SD 34 viral load in the non-vaccinated negative control (group 22) increased to about 4.1 log 10 c/μl and slightly decreased at SD41 to about 3.6.

The viral load in groups 18, 19 and 20 was between 1.3 and 1.8 log10 c/μl at SD 34 and between 1.6 and 2.1 at SD41.

PCV2 Viral Load in Fecal Swabs (qPCR Fecal Swab)

At SD19 (start of measurement), no viral load could be detected in all groups (log10 c/μl=0). At SD 34 viral load in the positive control (group 21) increased to about 1.5 log10 c/μl and further increased to about 2.2 at SD41.

The viral load in the non-vaccinated negative control (group 22) increased to about 3.6 log10 c/μl at SD34 and remained basically constant up to SD41.

The viral load in groups 18, 19 and 20 was in the range of the positive control group (between about 1.5 and 1.8 at SD34 and between about 1.8 and 2.2 at SD41).

Safety

No injection site reactions that were unacceptable were observed. Maximum average temperature increase was 0.4° C. at T=0+4 h. Thus, it could be shown that the inventive vaccine composition including the adjuvant combinations of squalane, vitamin E-acetate and silica gave acceptable results as regards vaccination efficacy without causing unacceptable site reactions.

### Example 7: Use of Different Pharma Grade Silica's to Constitute the Combination Vaccine

In this example various different types of silica's, all pharma grade (colloidal) amorphous silica's, are shown to be useful to produce a combination vaccine according to the invention. The method for preparing the vaccine was largely in line with Example 1, although for some vaccines the silica was added only after passage through the Microfluidizer™. This had no substantial impact on the final composition, all silica's were roughly distributed in the final composition with an average particle size of about 120 nm. However, when adding the silica after the passage through the Microfluidizer™, a very small percentage of the total volume of silica was present as large agglomerates (solid particles) above 10 μm (up to 200 μm). This however has no negative impact on the performance of the vaccine. The data for the various compositions are given in Table 12.

TABLE 12

| | | | | | Osmolality |
|---|---|---|---|---|---|
| | Microscopic | Mastersizer | | | mOsmol/ |
| Silica | view | in μm | Appearance | pH | kg |
| Aerosil | Solubilisate | 0.10 = 0.08 | Homo- | 7.41 | 214 |
| 200 | with particles | 0.50 = 0.12 | geneous, | | |
| | up to 3 μm | 0.90 = 0.17 | nearly white | | |
| | | 0.99 = 0.22 | | | |
| Aerosil | Solubilisate | 0.10 = 0.08 | Homo- | 7.42 | 189 |
| 200 | with | 0.50 = 0.12 | geneous, | | |
| | particles up | 0.90 = 0.22 | nearly white | | |
| | 3 μm, also | 0.99 = 66.96 | | | |
| | solid particles | | | | |
| | up to 50 μm | | | | |
| Aerosil | Solubilisate | 0.10 = 0.08 | Homo- | 7.38 | 212 |
| 200vv | with particles | 0.50 = 0.12 | geneous, | | |
| | up to 3 μm | 0.90 = 0.17 | nearly white | | |
| | | 0.99 = 0.23 | | | |
| Aerosil | Solubilisate | 0.10 = 0.08 | Homo- | 7.42 | 186 |
| 200vv | with | 0.50 = 0.12 | geneous, | | |
| | particles up | 0.90 = 13.10 | nearly white | | |
| | 3 μm, also | 0.99 = | | | |
| | solid particles | 143.79 | | | |
| | up to 200 μm | | | | |
| Aeroperl | Solubilisate | 0.10 = 0.08 | Homo- | 7.40 | 211 |
| 300 | with particles | 0.50 = 0.12 | geneous, | | |
| | up to 3 μm | 0.90 = 0.17 | nearly white | | |
| | | 0.99 = 0.22 | | | |
| Aeroperl | Solubilisate | 0.10 = 0.08 | Homo- | 7.42 | 187 |
| 300 | with | 0.50 = 0.12 | geneous, | | |
| | particles up | 0.90 = 0.18 | nearly white | | |
| | 3 μm, also | 0.99 = 63.65 | | | |
| | solid particles | | | | |
| | upto 100 μm | | | | |
| Aerosil | Solubilisate | 0.10 = 0.08 | Homo- | 7.40 | 211 |
| 380F | with particles | 0.50 = 0.12 | geneous, | | |
| | up to 3 μm | 0.90 = 0.17 | nearly white | | |
| | | 0.99 = 0.22 | | | |
| Aerosil | Solubilisate | 0.10 = 0.08 | Homo- | 7.41 | 186 |
| 380F | with | 0.50 = 0.13 | geneous, | | |
| | particles up | 0.90 = 25.44 | nearly white | | |
| | 3 μm, also | 0.99 = 85.02 | | | |
| | solid particles | | | | |
| | upto 100 μm | | | | |

Other silica's suitable for use in the present invention are Aerosil® 90, Aerosil® 130, Aerosil® 150, Aerosil® 200F, Aerosil® 255, Aerosil® OX 50, Aerosil® TT600 and Aeroperl® 300/30.

### CONCLUSIONS

The Examples 1 to 7 show that of the tested compositions only the novel adjuvant combination of squalane, vitamin E-acetate and silica gave acceptable results as regards vaccination efficacy against PCV2 and Mhyo infections without causing unacceptable site reactions. Hence, using this adjuvant composition, a combination vaccine can be provided against PCV2 and Mhyo using non-replicating immunogen of porcine circo virus type 2 and Mycoplasma hyopneumoniae for safe and effective intradermal administration.

1. A combination vaccine comprising a non-replicating immunogen of porcine circo virus type 2 and a non-replicating immunogen of Mycoplasma hyopneumoniae, characterized in that the combination vaccine is an oil-in-water emulsion comprising squalane, vitamin E-acetate and silica.

2. The combination vaccine according to claim 1, characterized in that it comprises squalane in an amount of 1 to 15% w/v.

3. The combination vaccine according to claim 1, characterized in that it comprises vitamin E-acetate in an amount of 2 to 20% w/v.

4. The combination vaccine according to claim 1, characterized in that it comprises an emulsifier with an HLB value of 8 to 20.

5. The combination vaccine according to claim 4, characterized in that the emulsifier is polysorbate 80.

6. The combination vaccine according to claim 4, characterized in that the emulsifier is present in an amount of 0.5 to 10% w/v.

7. The combination vaccine according to claim 1, characterized in that it comprises silica in an amount of 0.02 to 2% w/v.

8. The combination vaccine according to claim 1, characterised in that the non-replicating immunogen of porcine circo virus type 2 is recombinantly expressed protein encoded by the ORF2 gene of porcine circo virus type 2.

9. The combination vaccine according to claim 1, characterised in that the non-replicating immunogen of Mycoplasma hyopneumoniae comprises killed whole Mycoplasma hyopneumoniae.

10-12. (canceled)

13. A method of prophylactically treating an animal against an infection with porcine circo virus type 2 and an infection with Mycoplasma hyopneumoniae by administering into the dermis of the animal the combination vaccine according to claim 1.

14. An adjuvant composition for formulating a non-live vaccine, characterized in that the composition is an oil-in-water emulsion comprising squalane, vitamin E-acetate and silica.
